(19) United States
(12) Patent Application Publication
(10) Pub. No.: US 2011/0190583 A1
(43) Pub. Date: Aug. 4, 2011

(54) MEDICAL FIXED BALLOON, ACTUATOR FOR INTRADUCTAL MOVING BODY, AND ENDOSCOPE

(76) Inventors: Tsuyoshi ASHIDA, Ashigarakami-gun (JP); Takayuki Nakamura, Ashigarakami-gun (JP); Kuniaki Miyako, Ashigarakami-gun (JP); Shinichi Yamakawa, Ashigarakami-gun (JP); Yuya Morimoto, Ashigarakami-gun (JP)

(21) Appl. No.: 12/985,770

(22) Filed: Jan. 6, 2011

(30) Foreign Application Priority Data
Feb. 2, 2010 (JP) 2010-021544

(51) Int. Cl. A61B 1/00

(52) U.S. Cl. 600/115

(57) ABSTRACT

A medical fixed balloon disposed in an insertion portion to be inserted into a body cavity so as to fix the insertion portion to the inside of the body cavity, comprising:
an inflation membrane inflated by supplying a fluid into an inside of the inflation membrane and whose outer peripheral surface is brought into contact with an inner wall of the body cavity, the inflation membrane having regions extended with a predetermined extension rate and having a plurality of low extension regions of a lower extension rate than the predetermined extension rate, formed on a line segment from a base end to a distal end along an insertion axis of the insertion portion.

### BACKGROUND OF THE INVENTION

1. Field of the Invention

The present invention relates to a medical fixed balloon, an actuator for intraductal moving body, and an endoscope and particularly to a technology for fixing a medical instrument inserted into a body cavity to an inner wall of the body cavity.

2. Description of the Related Art

Insertion of an endoscope into a large intestine is extremely difficult since a large intestine has a meandering structure in the body and there is a portion not fixed to the body cavity. Thus, a lot of experiences are required for mastering the insertion procedure, and immature insertion procedure results in a great pain imparted to a patient.

A portion in the large intestine where insertion is considered to be particularly difficult is a sigmoid colon and a transverse colon. Unlike the other colons, the sigmoid colon and transverse colon are not fixed to the inside of the body cavity. Thus, they can take arbitrary forms in the body cavity within ranges of their own lengths and they also deform in the body cavity by a contact force when the endoscope is inserted.

In the insertion into a large intestine, in order to reduce contact with an intestinal tract during the insertion as much as possible, it is important to linearize the sigmoid colon or the transverse colon. There have been many procedures for the linearization proposed so far, and some insertion assisting tools that reduce a bending degree by hauling in the bending intestinal tract are proposed.

For example, Japanese Patent Application Laid-Open No. 11-9545 and Japanese Patent Application Laid-Open No. 2006-223895 disclose technologies in which four expandable/shrinkable variable tubes are wound into a spiral shape on the outer peripheral surface of a flexible pipe, and by changing the pressure in each variable tube so as to sequentially expand/shrink the four variable tubes, the outer peripheral surface of a shell expands/shrinks, and an expanded part is moved from the tip end side to the at-hand side so that an intestinal tract is hauled in.

However, a vertical motion of the plurality of variable tubes has little effect to move the contact face of the tube. Only if a fold of the intestinal tract efficiently enters a groove between the expanded tubes, the hauling effect can be exerted, but the sigmoid colon has little folds, and since the intestinal tract is linearized and a projecting amount of the fold is reduced in a hauling process, the hauling effect is extremely reduced.

On the other hand, when one balloon is inflated and a first portion on the outer peripheral surface of the balloon is brought into contact with and locked by an inner wall of an intestinal tract, for example, by moving the outer peripheral surface of the balloon along the inner wall of the intestinal tract to a second portion of the outer peripheral surface of the balloon continuing from the first portion, in a state in which the balloon is in contact with the inner wall of the intestinal tract, the inner wall of the intestinal tract can be hauled in with the movement from the first portion to the second portion, for example. However, since a living tissue such as an intestinal tract has a nature of expanding/shrinking not only in the tract diameter direction but also along the tract inner wall by elasticity of the tissue when a stress is applied and of returning to a state before the expansion/shrinkage by a restoring force by the elasticity when the stress is cancelled, if the balloon is made to shrink and is separated from the inner wall of the intestinal tract, the hauled-in inner wall of the intestinal tract returns to the original state by the above-described restoring force.

As described above, it is difficult to generate a locking force by a single balloon to be locked by an intestinal wall and to generate a propulsive force so as to relatively move the balloon with respect to the intestinal wall.

Then, such a propulsive mechanism of a method (rotating balloon method) that when two balloons are arranged side by side in the intraductal moving direction, for example, in which one of the balloons is made a locking (rotating) balloon and the other balloon as a driving balloon, the locking (rotating) balloon is inflated and locked by the intestinal tract and then, the driving balloon is inflated and controlled so as to press the locking (rotating) balloon and to rotate the locking (rotating) balloon has been examined. According to this propulsive mechanism, larger propulsive amount and propulsive force than the case of using only one balloon can be obtained, and the intraductal moving body can be relatively moved with respect to the intestinal wall reliably.

These balloons or particularly the locking balloon can fix an endoscopic insertion portion and the like to an inner wall of an intestinal tract by being attached to an insertion medical instrument such as the endoscopic insertion portion or a distal end portion of a catheter, for example, and inflated within the body cavity.

As a balloon for fixing an insertion medical instrument to an inner wall of an intestinal tract, a medical fixed balloon in which projecting and recessed portions which assist fixing is disposed in a portion in contact with the body cavity wall is proposed (Japanese Patent Application Laid-Open No. 2002-301020).

Also, as another balloon for fixing an insertion medical instrument to an inner wall of an intestinal tract, a balloon for medical tube characterized by having a expandable small portion formed by cross-link treatment at a part in the circumferential direction is proposed (Japanese Patent Application Laid-Open No. 11-405).

As shown by a Stribeck curve in FIG. 13, a lubrication state on a friction surface has generally four types of regions (I: clean surface, II: boundary lubrication, III: mixed lubrication, IV: fluid lubrication) (“Basic Tribology” by Hiromu Hashimoto, pp. 94)

However, in the body cavity, due to presence of a body fluid and flexibility of the balloon (inflatable body), lubricated contact is formed in the fluid lubrication region or the mixed lubrication region, and an influence of the body fluid on a body-cavity wall fixing force of the balloon is not considered at all in prior-art medical balloons such as those in Japanese Patent Application Laid-Open No. 2002-301020 and Japanese Patent Application Laid-Open No. 11-405. Particularly, a frictional force is hard to occur between the balloon and the inner wall of an intestinal tract due to the body fluid, and there is a problem that the body-cavity fixing force to the inner wall of the intestinal tract by the balloon is lowered.

Also, since the balloon is inflated not only in the circumferential direction of an endoscope but also in the axial direction when being inflated, there is a problem that a pressure applied to the balloon cannot be efficiently used for the body-cavity wall fixing force.

### SUMMARY OF THE INVENTION

The present invention was made in view of the above circumstances and has an object to provide a medical fixed balloon, an actuator for intraductal moving body, and an endoscope that eliminates the influence of the body fluid in the body cavity and can obtain a reliable and sufficient body-cavity fixing force to the inner wall of an intestinal tract.

In order to achieve the above objects, a medical fixed balloon according to a first aspect is a medical fixed balloon disposed in an insertion portion to be inserted into a body cavity so as to fix the insertion portion to the inside of the body cavity, comprising:

an inflation membrane inflated by supplying a fluid into an inside of the inflation membrane and whose outer peripheral surface is brought into contact with an inner wall of the body cavity, the inflation membrane having regions extended with a predetermined extension rate and having a plurality of low extension regions of a lower extension rate than the predetermined extension rate, formed on a line segment from a base end to a distal end along an insertion axis of the insertion portion.

With the medical fixed balloon according to the first aspect, since the inflation membrane has the plurality of low extension regions of a lower extension rate than the predetermined extension rate formed on the line segment from the base end to the distal end along the insertion axis of the insertion portion, the influence of the body fluid in the body cavity is eliminated, and the reliable and sufficient body-cavity fixing force to the inner wall of the intestinal tract can be obtained.

Also, by providing the low extension region along the insertion axis, the balloon can be easily inflated in the circumferential direction, and the pressure applied to the balloon can be efficiently used for the body-cavity wall fixing force.

As a medical fixed balloon according to a second aspect, in the medical fixed balloon according to the first aspect, the plurality of low extension regions are preferably formed point-symmetrically in a discrete manner on a section orthogonal to the insertion axis.

As a medical fixed balloon according to a third aspect, in the medical fixed balloon according to the first or second aspect, the low extension regions preferably have inflation regulated at least along the insertion axis of the inflation membrane.

As a medical fixed balloon according to a fourth aspect, in the medical fixed balloon according to the third aspect, the low extension regions preferably have predetermined extension rigidity in the direction of the insertion axis and has inflation of the inflation membrane along the insertion axis regulated by the predetermined extension rigidity.

As a medical fixed balloon according to a fifth aspect, in the medical fixed balloon according to any one of the first to fourth aspects, in the inflation membrane during inflation by the supply of the fluid, it is preferable that the region of the inflation membrane of the predetermined extension rate becomes a projection portion, the low extension regions become recess portions, the projection portion is brought into contact with the inner wall of the body cavity, and the recess portions fluidize the body fluid in the body cavity.

As a medical fixed balloon according to a sixth aspect, in the medical fixed balloon according to any one of the first to fifth aspects, the low extension regions are preferably formed by resin members or filamentous members along the insertion axis arranged in the inflation membrane.

An actuator for an intraductal moving body according to a seventh aspect has a first inflation/deflation member provided with a first portion that inflates and fills a gap between the intraductal moving body and a body-cavity ductal wall when the portion is brought into contact with the body-cavity ductal wall and a second portion that is brought into contact with the body-cavity ductal wall and generates a propulsive force, a part of the member being fixed to the intraductal moving body, a second inflation/deflation member fixed to the intraductal moving body and inflated and brought into contact with the body-cavity ductal wall, a driving inflation/deflation member that is arranged side by side with the first inflation/deflation member and the second inflation/deflation member in the intraductal moving direction and drives the first inflation/deflation member fixed to the intraductal moving body, and a control portion that executes control so that at least either one of the first inflation/deflation member and the second inflation/deflation member is inflated and held in a state locked by the body-cavity ductal wall, and relative positions of the intraductal moving body and the body-cavity ductal wall are changed so that the first portion of the first inflation/deflation member becomes the second portion by inflation/deflation driving of the driving inflation/deflation member, in which at least the first inflation/deflation member is a balloon that brings the outer peripheral surface of the inflation membrane of a predetermined extension rate into contact with the body-cavity ductal wall and fixes the insertion portion to the body-cavity ductal wall, and the inflation membrane has a plurality of low extension regions of a lower extension rate than the predetermined extension rate formed on a line segment from a base end to a distal end along the insertion axis of the insertion portion.

As an actuator for an intraductal moving body according to an eighth aspect, in the actuator for an intraductal moving body according to the seventh aspect, the second inflation/deflation member preferably has the plurality of low extension regions.

As an actuator for an intraductal moving body according to a ninth aspect, in the actuator for an intraductal moving body according to the seventh or eighth aspect, the plurality of low extension regions are preferably formed point-symmetrically in a discrete manner on a section orthogonal to the insertion axis.

An endoscope according to a tenth aspect includes a medical fixed balloon according to any one of the first to sixth aspects.

An endoscope according to an eleventh aspect includes an actuator for an intraductal moving body according to any one of the seventh to ninth aspects.

As described above, according to the present invention, such advantages can be obtained that the influence of the body fluid in the body cavity can be eliminated, and reliable and sufficient body-cavity fixing force to the inner wall of the intestinal tract can be obtained.

### BRIEF DESCRIPTION OF THE DRAWINGS

FIG. 1 is a configuration diagram illustrating a configuration of an electronic endoscope according to an embodiment of the present invention;

FIG. 2 is an enlarged sectional view of a distal end portion of an insertion portion of the electronic endoscope in FIG. 1;

FIG. 3 is a block configuration diagram of a balloon controller in FIG. 1 that controls pressures of first and second driving balloons, a locking balloon, and a holding balloon;

FIG. 4 is a time chart of a forward moving operation, which is a propulsive operation by the balloon controller in FIG. 3;

FIGS. 5A to 5F are outline sectional views illustrating inflation and deflation of each balloon in accordance with the time chart of the forward moving operation shown in FIG. 4;

FIG. 6 is a time chart of a backward moving operation, which is the propulsive operation by the balloon controller in FIG. 3;

FIGS. 7A to 7F are outline sectional views illustrating inflation and deflation of each balloon in accordance with the time chart of the backward moving operation shown in FIG. 6;

FIG. 8 is a diagram illustrating an appearance of the locking balloon in FIG. 2 in a deflated state fixed to the distal end of the insertion portion;

FIG. 9 is a diagram illustrating transition from the deflation of the locking balloon in FIG. 8 to inflation;

FIG. 10 is a diagram illustrating a section of the locking balloon crossing the insertion axis at a right angle in the inflation transition in FIG. 9;

FIG. 11 is a diagram illustrating a section of the locking balloon in the insertion axis direction in the inflation transition in FIG. 9;

FIG. 12 is a diagram illustrating a variation of the locking balloon in FIG. 8; and

FIG. 13 is a graph of Stribeck curve illustrating lubrication states of a friction surface.

### DETAILED DESCRIPTION OF THE PREFERRED EMBODIMENTS

A medical fixed balloon, an actuator for an intraductal moving body, and an endoscope according to the present invention will be described below in detail referring to the attached drawings.

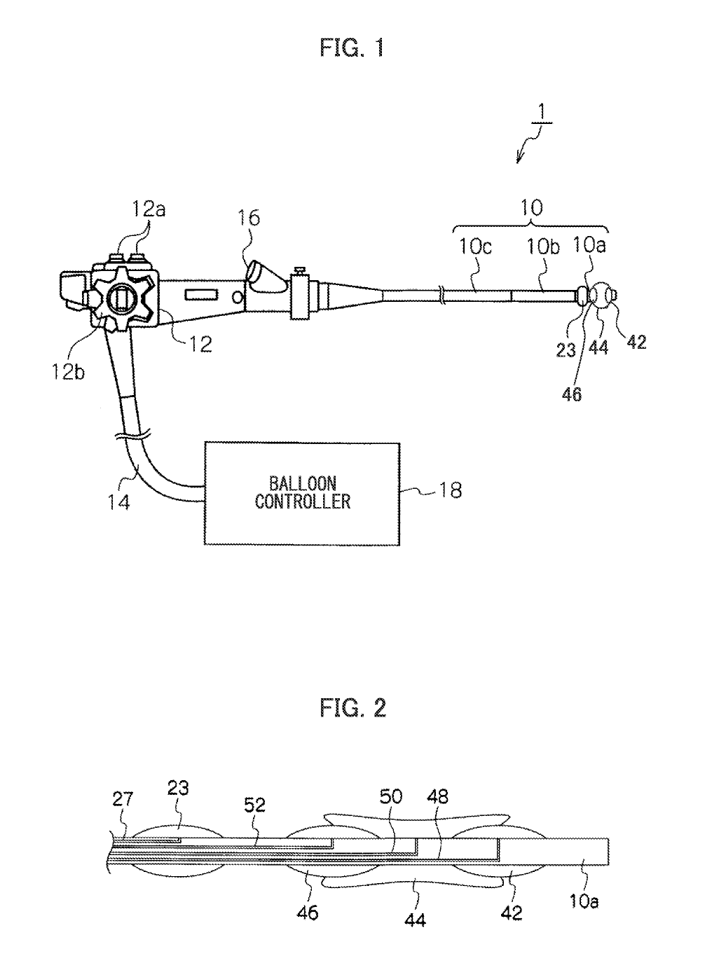

FIG. 1 is a configuration diagram illustrating a configuration of an electronic endoscope according to an embodiment of the present invention.

As shown in FIG. 1, an electronic endoscope 1 of this embodiment includes an insertion portion 10, which is an intraductal moving body, inserted into a duct of a subject body and moving through the duct and an operation portion 12 disposed consecutively to a base end portion of the insertion portion 10. In a distal end portion 10a disposed consecutively to the distal end of the insertion portion 10, an objective lens that takes in image light of a portion to be observed in the subject body and an image pickup element that picks up the image light (neither of them is shown) are incorporated. An image inside the subject body taken by the image pickup element is displayed as an endoscopic image on a monitor of a processor connected to a cord 14 (neither of them is shown).

Also, in the distal end portion 10a, an illumination window that radiates illumination light from a light source device (not shown) to the portion to be observed, a forceps outlet communicating with a forceps inlet 16, a nozzle through which washing water or air for washing off stains on an observation window that protects the objective lens is injected by operating an air/water feeding button 12a and the like are disposed.

In the rear of the distal end portion 10a, a bent portion 10b in which a plurality of bent pieces are connected is disposed. The bent portion 10b is bent and operated vertically and horizontally when an angle knob 12b disposed on the operation portion 12 is operated and a wire inserted through the insertion portion 10 is pushed/pulled. As a result, the distal end portion 10a is directed to a desired direction in the subject body.

In the rear of the bent portion 10b, a flexible portion 10c having flexibility is disposed. The flexible portion 10c has a length of 1 to several m so that the distal end portion 10a can reach the portion to be observed, and a distance from a patient is kept to such a degree that grasping and operation of the operation portion 12 by an operator is not interfered.

In the distal end portion 10a, a first driving balloon 42 as a driving inflation/deflation member, a second driving balloon 46 as a third inflation/deflation member, and a locking balloon 44 as a first inflation/deflation member, which will be described later, arranged side by side in the advance direction moving through the duct and as fixed inflation/deflation members are attached. The first driving balloon 42, the second driving balloon 46, and the locking balloon 44 are mainly made of latex rubber capable of inflation/deflation and are connected to a balloon controller 18 that controls pressures inside the balloons.

The first driving balloon 42 and the locking balloon 44 as well as the locking balloon 44 and the second driving balloon 46 are arranged adjacently to each other in the distal end portion 10a and formed on the entire circumference in the circumferential direction of the insertion portion 10. Also, the first driving balloon 42, the second driving balloon 46, and the locking balloon 44 may be arranged symmetrically in the uniform shape in the circumferential direction of the insertion portion 10 or does not have to be symmetric nor in the uniform shape in the circumferential direction of the insertion portion 10.

Also, the first driving balloon 42, the second driving balloon 46, and the locking balloon 44 may be arranged in the bent portion 10b or the flexible portion 10c.

With the electronic endoscope 1 constituted as above, if an inner wall surface of a duct bent in a complicated way such as a large intestine or a small intestine is to be observed, the insertion portion 10 is inserted into a subject body in a state in which the first driving balloon 42, the second driving balloon 46, and the locking balloon 44 are deflated, a light source device is lighted so as to illuminate the inside of the subject body, and an endoscopic image obtained by the image pickup element is observed on a monitor.

If the distal end portion 10a reaches the duct, inflation/deflation of the first driving balloon 42, the second driving balloon 46, and the locking balloon 44 is controlled by a balloon controller 18, and a pressing force is applied to the inner wall surface of the duct. As a result, the inner wall surface of the duct is hauled in, and the insertion portion 10 is propelled relatively forward or backward in the advance direction with respect to the inner wall surface of the duct.

Detailed explanation of the flow of the propulsive operation will be made later. Also, in the following explanation, an operation in which the distal end portion 10a is propelled forward in the advance direction is referred to as a forward moving operation, while an operation in which the distal end portion 10a is propelled backward in the advance direction is referred to as a backward moving operation.

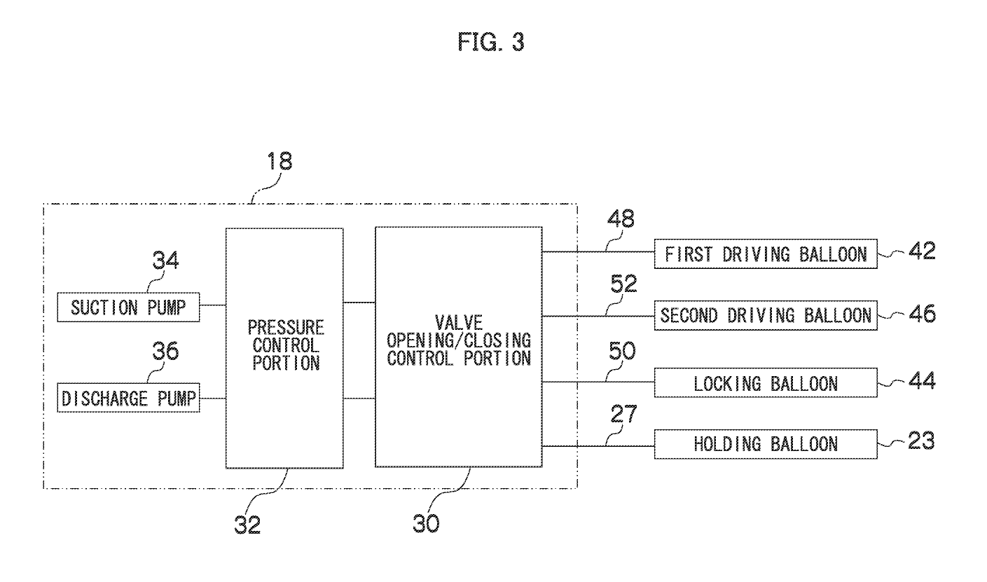

Subsequently, an actuator for an intraductal moving body of this embodiment composed of the first driving balloon 42 and the locking balloon 44 as well as the locking balloon 44 and the second driving balloon 46 will be described referring to FIGS. 2 and 3.

FIG. 2 is an enlarged sectional view of the distal end portion 10a of the insertion portion 10 in this embodiment. As shown in FIG. 2, in this embodiment, the three balloons, that is, the first driving balloon 42, the locking balloon 44, and the second driving balloon 46 are fastened and disposed on the outer peripheral surface of the distal end portion 10a, respectively, by a bobbin or the like in order from the front in the advance direction (the direction of the distal end along the longitudinal axis of the insertion portion 10) in the distal end portion 10a of the insertion portion 10.

Also, when the locking balloon 44 is not in contact with the ductal wall, a holding balloon 23 as a second inflation/deflation member that holds the position of the distal end portion 10a of the insertion portion 10 is also fastened and disposed on the outer peripheral surface of the distal end portion 10a by a bobbin or the like. In the propulsive operation, at least either one of the locking balloon 44 and the holding balloon 23 is inflated and brought into contact with the ductal wall and locked thereby. In this embodiment, the medical fixed balloon is composed of the locking balloon 44.

These first driving balloon 42, the second driving balloon 46, the locking balloon 44, and the holding balloon 23 are all entirely made of latex rubber capable of inflation/deflation, and a section orthogonal to the longitudinal axis (insertion axis) of the insertion portion 10 forms a doughnut shape (not shown) around the longitudinal axis (insertion axis).

The locking balloon 44 is a balloon having an inflation characteristic that can be brought into contact with the inner wall surface of the ductal wall and locked thereby during inflation, while the first driving balloon 42 and the second driving balloon 46 are balloons having an inflation characteristic not brought into contact with the inner wall surface of the ductal wall as long as the distal end portion 10a is located substantially at the center position of the section of the duct even during the inflation.

Also, it is preferable that the first driving balloon 42, the second driving balloon 46, and the locking balloon 44 are different in the shape from each other.

The holding balloon 23, the first driving balloon 42, the second driving balloon 46, and the locking balloon 44 are fastened to the outer peripheral surface of the distal end portion 10a of the insertion portion 10 by a bobbin or the like, and the outer peripheral portion is constituted capable of inflation/deflation in the radial direction of the distal end portion 10a of the insertion portion 10.

In this embodiment, the actuator for an intraductal moving body is constituted by arranging the first driving balloon 42, the locking balloon 44, the second driving balloon 46, and the holding balloon 23 in the order from the front in the intraductal moving direction, but it may be so constituted by arranging the holding balloon 23, the first driving balloon 42, the locking balloon 44, and the second driving balloon 46 in the order from the front in the intraductal moving direction.

As shown in FIG. 3, the balloon controller 18 is constituted to include a valve opening/closing control portion 30 and a pressure control portion 32 that can adjust internal pressures of the first driving balloon 42, the second driving balloon 46, the locking balloon 44, and the holding balloon 23 independently of each other.

Then, in the balloon controller 18, the first driving balloon 42, the second driving balloon 46, the locking balloon 44, and the holding balloon 23 are connected to a suction pump 34 and a discharge pump 36 through the valve opening/closing control portion 30 and the pressure control portion 32.

Inside the distal end portion 10a, an air feeding pipe 48 communicating with the first driving balloon 42 and through which gas is fed, an air feeding pipe 50 communicating with the locking balloon 44 and through which gas is fed, an air feeding pipe 52 communicating with the second driving balloon 46 and through which gas is fed, and an air feeding pipe 27 communicating with the holding balloon 23 and through which gas is fed are disposed (See FIG. 2). These air feeding pipes 48, 50, 52, and 27 are connected to the balloon controller 18 passing through bent portion 10b and the flexible portion 10c and the cord 14 (See FIG. 1).

The flow of the propulsive operation, which will be described later, is executed by controlling opening/closing of a valve (not shown) connected to each balloon by the valve opening/closing control portion 30 and by controlling the suction pump 34 and the discharge pump 36 by the pressure control portion 32.

### <Flow of Propulsive Operation>

### “Forward Moving Operation”

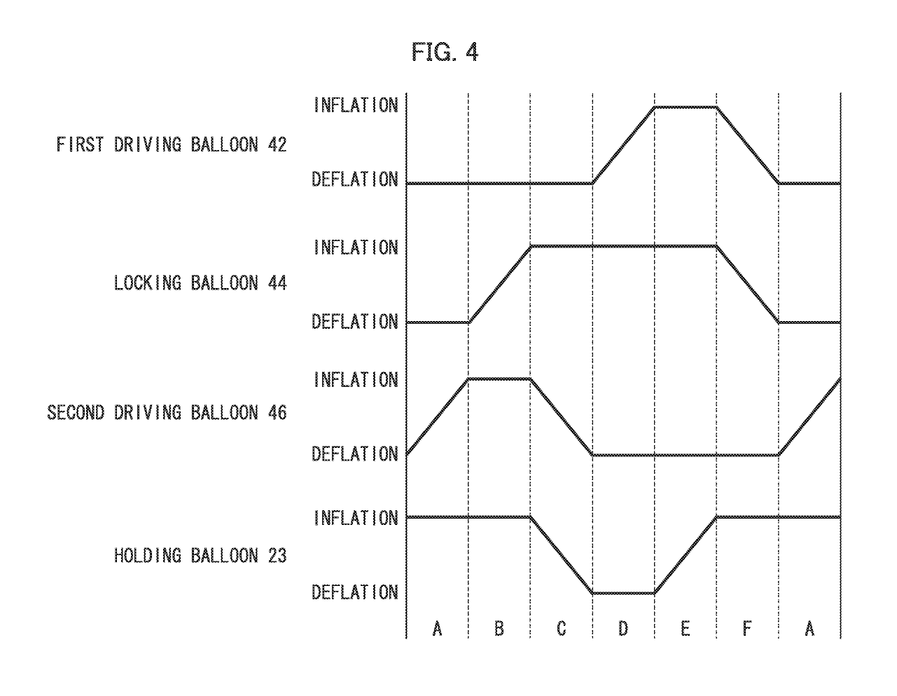

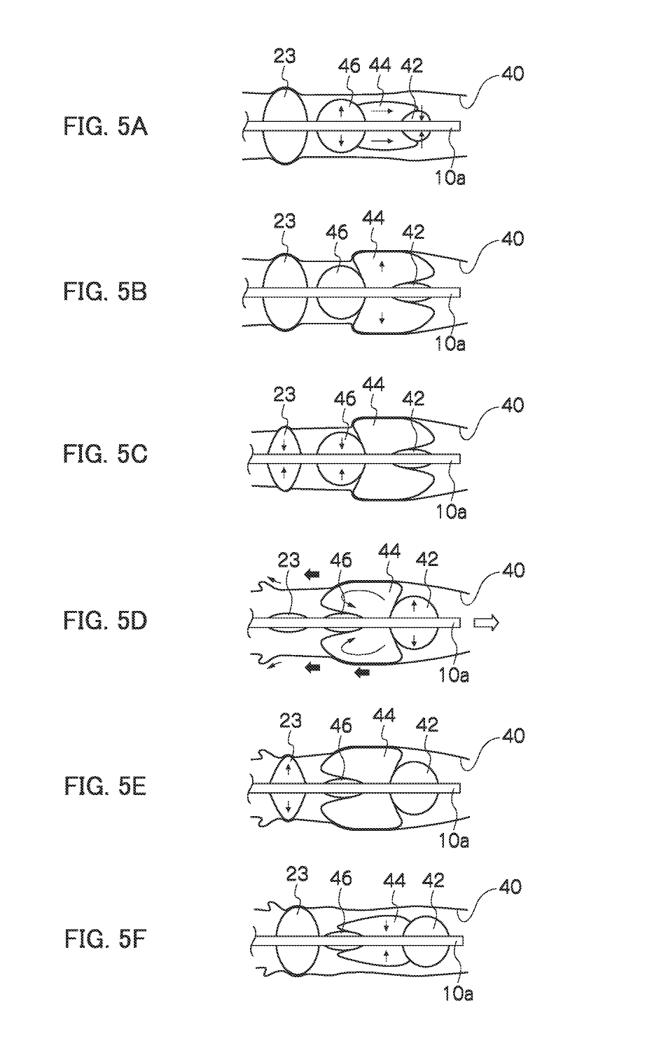

Subsequently, the forward moving operation in the propulsive operation in this embodiment will be described referring to FIGS. 4 and 5.

FIG. 4 is a time chart of the forward moving operation in the propulsive operation. Also, FIGS. 5A to 5F are outline sectional views illustrating states of inflation and deflation of each balloon in correspondence with the time chart shown in FIG. 4.

First, in a state in which the first driving balloon 42, the locking balloon 44, and the second driving balloon 46 are all deflated, consider a state in which the distal end portion 10a of the electronic endoscope 1 is inserted into a measurement target (here, a large intestine, for example). At this time, the holding balloon 23 is inflated and locked by an intestinal wall 40 as the body-cavity ductal wall.

Then, from the state in which the holding balloon 23 is inflated and locked by the intestinal wall 40 is held and the first driving balloon 42, the locking balloon 44, and the second driving balloon 46 are all deflated, gas is filled in the second driving balloon 46 so as to inflate the balloon (process A in FIG. 4). The state of the inflation of the balloon at this time can be depicted as in FIG. 5A. As shown in FIG. 5A, by means of the inflation of the second driving balloon 46, the locking balloon 44 is pushed out to the side of the first driving balloon 42 and flops over the first driving balloon 42. Subsequently, the gas is filled in the locking balloon 44 so as to inflate the balloon and the locking balloon 44 is locked by the intestinal wall 40 (process B in FIG. 4). The state of the inflation and deflation of the balloon at this time can be depicted as in FIG. 5B.

Also, here, in the locking balloon 44, when the balloon is inflated and brought into contact with the intestinal wall 40, a portion filling the gap between the insertion portion 10 and the intestinal wall 40 is considered as a first portion, and a portion in contact with the intestinal wall 40 is considered as a second portion.

Subsequently, the gas is suctioned from the holding balloon 23 and the second driving balloon 46 so as to deflate them (process C in FIG. 4). The state of the deflation of the balloon at this time can be depicted as in FIG. 5C.

Then, the gas is filled in the first driving balloon 42 so as to inflate the balloon (process D in FIG. 4). The state of the inflation of the balloon at this time can be depicted as in FIG. 5D.

As shown in FIG. 5D, by gradually inflating the first driving balloon 42, the first driving balloon 42 gradually presses the locking balloon 44. Moreover, since the second driving balloon 46 is gradually deflated, the locking balloon 44 is pushed so as to be sequentially fed out toward the rear in the advance direction of the distal end portion 10a in a state in which the surface thereof is in contact with the intestinal wall 40, or the balloon is pushed so that the surface is moved. Also, as described above, if the locking balloon 44 is considered to be provided with the first portion and the second portion, it can be considered that a part of the first portion on the intestinal wall 40 side on the front side in the advance direction of the distal end portion 10a is brought into contact with the intestinal wall 40 and pushed so as to become the second portion. As a result, the locking balloon 44 imparts a pressing force to the intestinal wall 40 rearward in the advance direction of the distal end portion 10a (black arrows in FIG. 5D).

That is, the locking balloon 44 is fed out rearward in the advance direction of the distal end portion 10a in contact with the intestinal wall 40, like a so-called caterpillar (registered trademark) (caterpillar track).

Thus, the intestinal wall 40 is hauled in rearward in the advance direction of the distal end portion 10a. Therefore, as shown by a white arrow in FIG. 5D, the distal end portion 10a of the electronic endoscope 1 is relatively propelled (moved forward) to the front in the advance direction with respect to the intestinal wall 40.

Subsequently, the holding balloon 23 is filled with the gas and inflated and locked by the intestinal wall 40 (process E in FIG. 4). The state of the inflation of the balloon at this time can be depicted as in FIG. 5E.

Subsequently, the state in which the holding balloon 23 is inflated and locked by the intestinal wall 40 is held, and the first driving balloon 42 and the locking balloon 44 are deflated by suctioning the gas (process F in FIG. 4). The state of the deflation of the balloon at this time can be depicted as in FIG. 5F.

Subsequently, by filling the gas in the second driving balloon 46 so as to inflate the balloon (process A in FIG. 4), the state is returned to the one shown in FIG. 5A.

After that, if the forward moving operation is to be continued, the process A to the process F in FIG. 4 are repeated.

### “Backward Moving Operation”

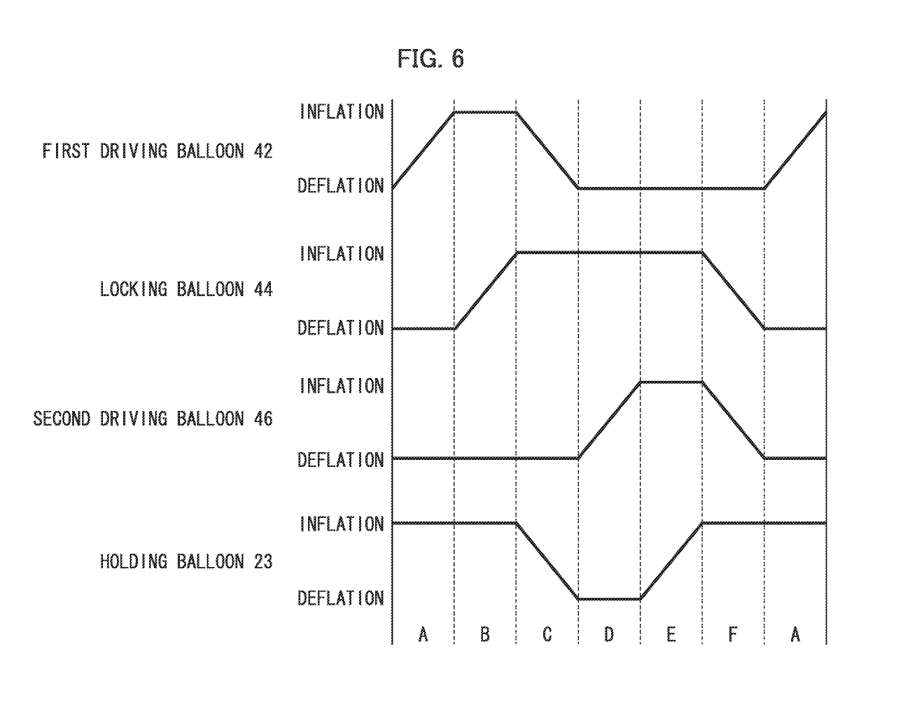

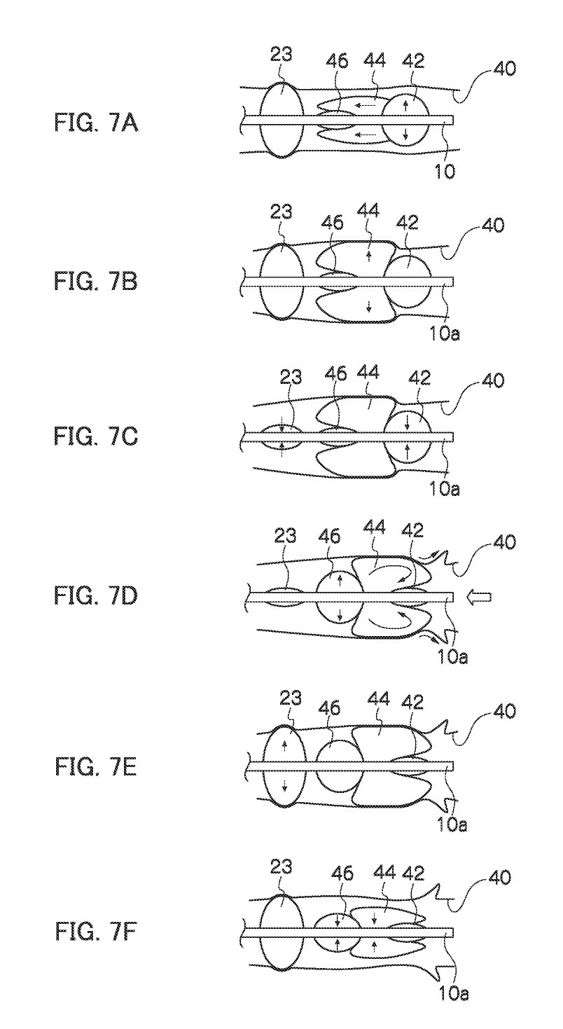

Subsequently, the backward moving operation in the propulsive operation in this embodiment will be described referring to FIGS. 6 and 7A to 7F.

FIG. 6 is a time chart of the backward moving operation in the propulsive operation. Also, FIGS. 7A to 7F are outline sectional views illustrating states of inflation and deflation of each balloon in correspondence with the time chart shown in FIG. 6.

First, in a state in which the first driving balloon 42, the locking balloon 44, and the second driving balloon 46 are all deflated, consider a state in which the distal end portion 10a of the electronic endoscope 1 is inserted into a measurement target (here, a large intestine, for example). At this time, the holding balloon 23 is inflated and locked by an intestinal wall 40.

Then, the state in which the locking balloon 44 and the second driving balloon 46 are deflated is held, and the gas is filled in the first driving balloon 42 so as to inflate the balloon (process A in FIG. 6). The state of the inflation of the balloon at this time can be depicted as in FIG. 7A. As shown in FIG. 7A, by means of the inflation of the first driving balloon 42, the locking balloon 44 is pushed out to the side of the second driving balloon 46 and flops over the second driving balloon 46.

Subsequently, the gas is filled in the locking balloon 44 so as to inflate the balloon and the locking balloon 44 is locked by the intestinal wall 40 (process B in FIG. 6). The state of the inflation of the balloon at this time can be depicted as in FIG. 7B. Also, here, in the locking balloon 44, a portion filling the gap between the insertion portion 10 and the intestinal wall 40 when the balloon is brought into contact with the intestinal wall 40 is considered as a first portion, and a portion in contact with the intestinal wall 40 is considered as a second portion.

Subsequently, the gas is suctioned from the holding balloon 23 and the first driving balloon 42 so as to deflate them (process C in FIG. 6). The state of the deflation of the balloon at this time can be depicted as in FIG. 7C.

Then, the gas is filled in the second driving balloon 46 so as to inflate the balloon (process D in FIG. 6). The state of the inflation of the balloon at this time can be depicted as in FIG. 7D.

As shown in FIG. 7D, by gradually inflating the second driving balloon 46, the second driving balloon 46 gradually presses the locking balloon 44. Moreover, the locking balloon 44 is pushed so that the surface thereof is sequentially fed out toward the front in the advance direction of the distal end portion 10a or pushed so as to move the surface. Also, as described above, if the locking balloon 44 is considered to be provided with the first portion and the second portion, it can be considered that a part of the first portion on the intestinal wall 40 side on the rear side in the advance direction of the distal end portion 10a is brought into contact with the intestinal wall 40 and pushed so as to become the second portion. As a result, the locking balloon 44 imparts a pressing force to the intestinal wall 40 to the front in the advance direction of the distal end portion 10a (black arrows in FIG. 7D).

That is, the locking balloon 44 is fed out toward the front in the advance direction of the distal end portion 10a in contact with the intestinal wall 40, like a so-called caterpillar (registered trademark) (caterpillar track).

Thus, the intestinal wall 40 is hauled in to the front in the advance direction of the distal end portion 10a. Therefore, as shown by a white arrow in FIG. 7D, the distal end portion 10a of the electronic endoscope 1 is relatively propelled to the rear in the advance direction (moved backward) with respect to the intestinal wall 40.

Subsequently, the holding balloon 23 is filled with the gas and inflated and locked by the intestinal wall 40 (process E in FIG. 6). The state of the inflation of the balloons at this time can be depicted as in FIG. 7E.

Subsequently, the gas is suctioned from the locking balloon 44 and the second driving balloon 46 so as to deflate the balloons (process F in FIG. 6). The state of the deflation of the balloons can be depicted as in FIG. 7F.

After that, if the backward moving operation is to be continued, the process A to the process F in FIG. 6 are repeated.

Subsequently, the locking balloon 44 (See FIG. 2) as the medical fixed balloon in this embodiment will be described in detail.

The locking balloon 44 is described to be composed of latex rubber capable of inflation/deflation, but not limited to that, and the locking balloon 44 may be composed of rubber or plastic elastomer or natural rubber, urethane rubber or silicone rubber as more preferable materials.

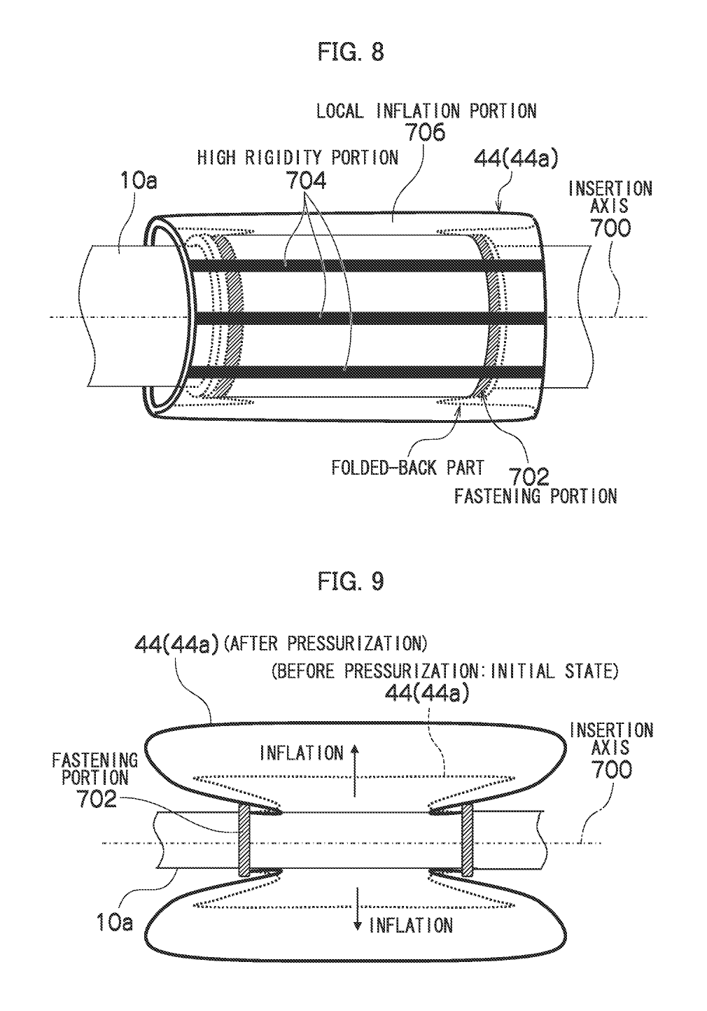

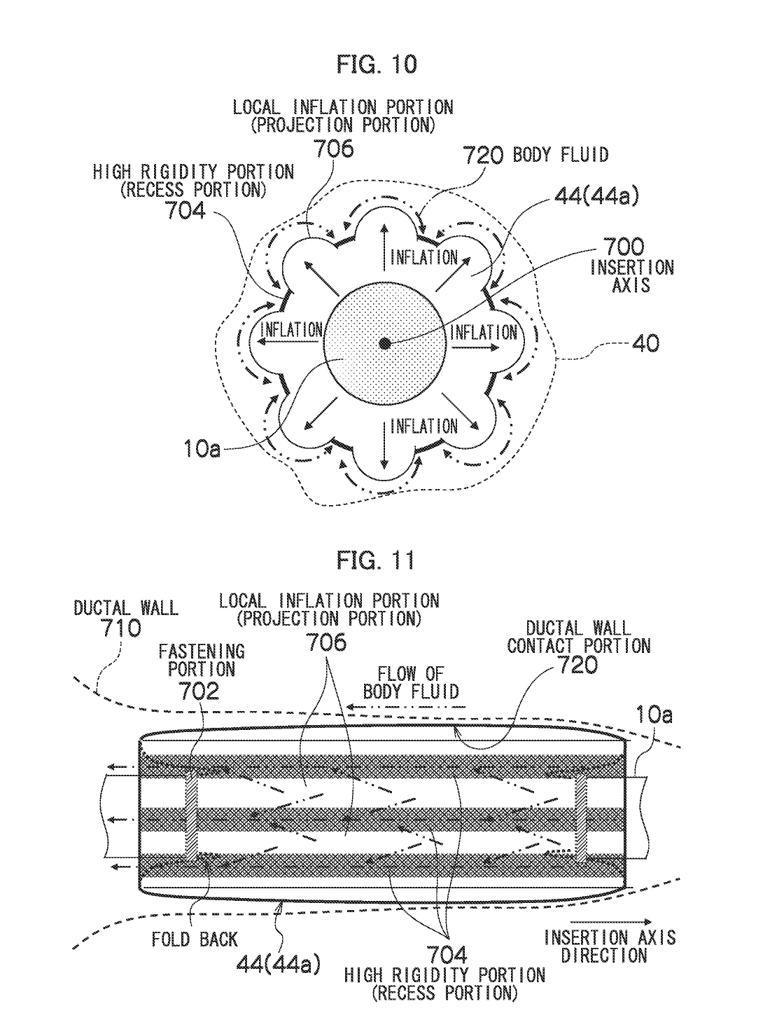

FIG. 8 is a diagram illustrating an appearance of the locking balloon in the deflated state, fastened to the distal end portion of the insertion portion. Also, FIG. 9 is a diagram illustrating transition from deflation to inflation of the locking balloon in FIG. 8. Moreover, FIG. 10 is a diagram illustrating a section of the locking balloon orthogonal to the insertion axis in the inflation transition in FIG. 9, and FIG. 11 is a diagram illustrating a section of the locking balloon in the insertion axis direction in the inflation transition in FIG. 9.

The locking balloon 44 as the medical fixed balloon has, as shown in FIG. 8, a cylindrical shape made of latex rubber capable of inflation/deflation, for example, and front and rear ends of the cylinder is fastened and fixed at a predetermined position of the distal end portion 10a of the insertion portion 10 by a fastening portion 702 such as a bobbin or the like in a state folded back to the outer peripheral surface of the distal end portion 10a.

Also, on the locking balloon 44, a plurality of high rigidity portions 704 are arranged in the band shape that reach the front and rear ends on the outer peripheral surface in a folded back state at the fastening portion 702 on a line segment along the insertion axis 700 of the insertion portion 10 in an inflation membrane 44a constituting the locking balloon 44 and have low extensibility and high rigidity. The region of the inflation membrane 44a without the high rigidity portion 704 is a band-shaped local inflation portion 706 having predetermined extensibility (higher extensibility than the high rigidity portion 704).

In this embodiment, the high rigidity portion 704 is composed of a resin or a filamentous member embedded in the inflation membrane 44a constituting the locking balloon 44, for example, but the high rigidity portion 704 (resin or filamentous member) may be installed on the surface of the inflation membrane 44a. The locking balloon 44 configured as above is, as shown in FIG. 9, inflated by the balloon controller 18 through the air feeding pipe 50.

By means of this inflation, as shown in FIG. 10, the locking balloon 44 has extension (inflation) suppressed in the high rigidity portion 704 with low extensibility and is locally inflated in the local inflation portion 706 having the predetermined extensibility (higher extensibility than the high rigidity portion 704). As a result, the outer surface of the locking balloon 44 has a recess portion formed by the high rigidity portion 704 and a projection portion formed by the local inflation portion 706.

Since the locking balloon 44 of this embodiment has the plurality of high rigidity portions 704 point-symmetrically with respect to the insertion axis of the insertion portion 10 as shown in FIG. 10, the projections and recesses of the high rigidity portions 704 and the local inflation portions 706 are also formed point-symmetrically with respect to the insertion axis of the insertion portion 10. That is, the high rigidity portions 704 need to be disposed at three locations or more and are preferably arranged substantially with point symmetry.

With the locking balloon 44 of this embodiment constituted as above, body fluid located in the local inflation portion 706 forming the projection portion is moved (fluidized) to the high rigidity portion 704 forming the recess portion.

As a result, as shown in FIG. 11, the locking balloon 44 of this embodiment forms a groove (recess portion) by the high rigidity portion on the outer surface by the inflation, and by fluidizing the body fluid adhering to the surface of the local inflation portion 706 to the groove (recess portion) by the high rigidity portion 704 so as to reduce the adhesion amount of the body fluid, the locking balloon 44 and the intestinal wall 40 are brought into contact with each other in the boundary lubrication region of the Stribeck curve (See FIG. 13).

As described above, in this embodiment, by bringing the local inflation portion 706 into contact with the intestinal wall 40 and pressing the wall in the boundary lubrication region of the Stribeck curve, the locking balloon 44 can obtain a sufficient fixing force (holding force) even under the body fluid.

Also, in this embodiment, since the high rigidity portion 704 is formed along the insertion axis 700 and a high rigidity portion is formed in the locking balloon 44 in the insertion axis 700 direction, the balloon is hard to be inflated in the insertion axis 700 direction and is easily inflated in the circumferential direction, and the balloon (locking balloon 44) can be fixed to the body cavity wall (intestinal wall 40) with a low internal pressure.

That is, according to this embodiment, when the distal end portion 10a of the insertion portion 10 of the electronic endoscope 1 is to be fixed to the body cavity, unique actions/effects as follows can be obtained:

(A1) the body fluid is discharged through the grooves of projections and recesses formed by a difference in the extensibility between the local inflation portions 706 and the high rigidity portions 704, and the locking balloon 44 can maintain the fixing force to the body-cavity wall (intestinal wall 40); and
(A2) by forming a high rigidity portion in a part in the circumferential direction, the balloon is hard to be inflated in the insertion axis direction and is easily inflated in the circumferential direction during the pressurization, and the locking balloon 44 can be efficiently fixed to the body-cavity wall (intestinal wall 40) located in the circumferential direction.

Moreover, the locking balloon 44 is rotated in the axial direction when propelling through the body cavity wall, but the balloon usually slips on the body-cavity wall surface during the axial rotation, which has made efficient propelling through the body-cavity wall difficult. On the other hand, according to the embodiment of the present invention,

(B1) the body fluid is discharged through the grooves of projections and recesses formed by a difference in the extensibility between the local inflation portions 706 and the high rigidity portions 704, and the locking balloon 44 can maintain the fixing force to the body-cavity wall (intestinal wall 40); and
(B2) by forming a high rigidity portion (high rigidity portion 704) in a part in the circumferential direction, the balloon is hard to be inflated in the insertion axis direction and is easily inflated in the circumferential direction during the pressurization, and the locking balloon 44 can be efficiently fixed to the body-cavity wall (intestinal wall 40) located in the circumferential direction.

By the effects of the above (B1) and (B2), the body-cavity wall (intestinal wall 40) can be fixed to the locking balloon 44 even during the rotary motion of the locking balloon 44, and a stable propulsive force can be generated. In this embodiment, the locking balloon 44 was described as an example, but the high rigidity portion 704 may be disposed in the holding balloon 23 as described above.

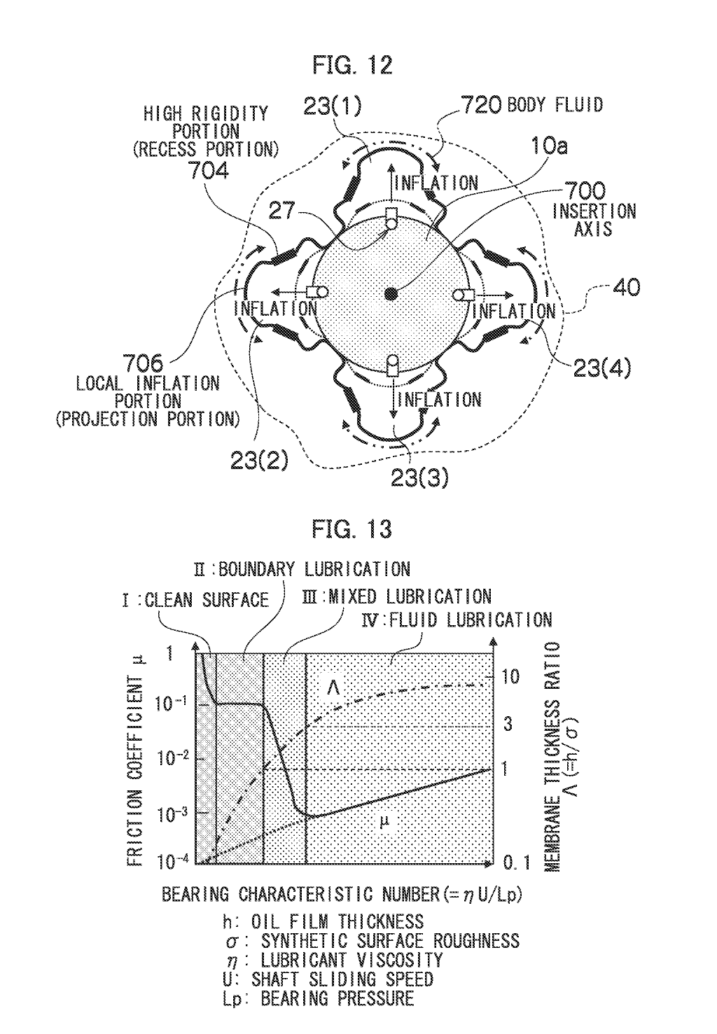

As described above, the locking balloon 44 of this embodiment is constituted by a single cylindrical balloon in the shape of a doughnut around the insertion axis, but not limited to that, as shown in FIG. 12, which is a variation of the locking balloon 44 of FIG. 8, it may be configured by a plurality of, or four locking balloons 44(1) to (4), for example, arranged point-symmetrically to the insertion axis 700 on the outer periphery of the distal end portion 10a of the insertion portion 10 and may be configured such that by disposing the high rigidity portion 704 on each of the locking balloons 44(1) to (4), each local inflation portion 706 is brought into contact with the intestinal wall 40 so as to press the intestinal wall. In this case, it is only necessary that the high rigidity portion 704 is arranged symmetrically with respect to a radial line segment from the insertion axis 700.

Also, each of the locking balloons 44(1) to (4) is controlled by the balloon controller 18 to be inflated or deflated at the same timing, it flops over the first driving balloon 42 or the second driving balloon 46, and each is fed out rearward or forward in the advance direction of the distal end portion 10a while being in contact with the intestinal wall 40 like a caterpillar (registered trademark) (caterpillar track).

In the above explanation, the actuator for an intraductal moving body composed of the medical fixed balloon using the locking balloon 44 as an example, the first driving balloon 42, the holding balloon 23, and the second driving balloon 46, and an endoscope using the electronic endoscope 1 provided with the actuator for an intraductal moving body at the distal end portion 10a of the insertion portion 10 as an example are described, but the present invention can be applied also to a medical instrument provided with the medical fixed balloon of this embodiment. For example, a medical instrument to which the present invention can be applied includes a known dilation balloon catheter, a double-balloon endoscope and the like.

The medical fixed balloon, the actuator for an intraductal moving body, and the endoscope of the present invention have been described above in detail, but the present invention is not limited to the above examples, but it is needless to say that various improvements or deformation can be made within a range not departing from the gist of the present invention.

What is claimed is:

1. A medical fixed balloon disposed in an insertion portion to be inserted into a body cavity so as to fix the insertion portion to the inside of the body cavity, comprising:
an inflation membrane inflated by supplying a fluid into an inside of the inflation membrane and whose outer peripheral surface is brought into contact with an inner wall of the body cavity, the inflation membrane having regions extended with a predetermined extension rate and having a plurality of low extension regions of a lower extension rate than the predetermined extension rate, formed on a line segment from a base end to a distal end along an insertion axis of the insertion portion.

2. The medical fixed balloon according to claim 1, wherein
the plurality of low extension regions are formed point-symmetrically in a discrete manner on a section orthogonal to the insertion axis.

3. The medical fixed balloon according to claim 1, wherein
the low extension regions have inflation regulated at least along the insertion axis of the inflation membrane.

4. The medical fixed balloon according to claim 3, wherein
the low extension regions have a predetermined extension rigidity in the direction of the insertion axis and have inflation of the inflation membrane along the insertion axis regulated by the predetermined extension rigidity.

5. The medical fixed balloon according to claim 1, wherein
in the inflation membrane during inflation by the supply of the fluid, the region of the inflation membrane of the predetermined extension rate becomes a projection portion, the low extension regions become recess portions, the projection portion is brought into contact with the inner wall of the body cavity, and the recess portions fluidize the body fluid in the body cavity.

6. The medical fixed balloon according to claim 1, wherein
the low extension regions are formed by resin members or filamentous members along the insertion axis arranged in the inflation membrane.

7. An actuator for an intraductal moving body, comprising:
a first inflation/deflation member provided with a first portion that inflates and fills a gap between the intraductal moving body and a body-cavity ductal wall when the portion is brought into contact with the body-cavity ductal wall and a second portion that is brought into contact with the body-cavity ductal wall and generates a propulsive force, a part of which is fixed to the intraductal moving body;
a second inflation/deflation member fixed to the intraductal moving body and inflated and brought into contact with the body-cavity ductal wall;
a driving inflation/deflation member that is arranged side by side with the first inflation/deflation member and the second inflation/deflation member in the intraductal moving direction and drives the first inflation/deflation member fixed to the intraductal moving body; and
a control portion that executes control so that at least either one of the first inflation/deflation member and the second inflation/deflation member is inflated and held in a state locked by the body-cavity ductal wall and relative positions of the intraductal moving body and the body-cavity ductal wall are changed so that the first portion of the first inflation/deflation member becomes the second portion by inflation/deflation driving of the driving inflation/deflation member, wherein
at least the first inflation/deflation member is a balloon that brings the outer peripheral surface of the inflation membrane of a predetermined extension rate into contact with the body-cavity ductal wall and fixes the insertion portion to the body-cavity ductal wall, and the inflation membrane has a plurality of low extension regions of a lower extension rate than the predetermined extension rate formed on a line segment from a base end to a distal end along the insertion axis of the insertion portion.

8. The actuator for an intraductal moving body according to claim 7, wherein the second inflation/deflation member has the plurality of low extension regions.

9. The actuator for an intraductal moving body according to claim 7, wherein the plurality of low extension regions are formed point-symmetrically in a discrete manner on a section orthogonal to the insertion axis.

10. An endoscope, comprising a medical fixed balloon according to claim 1.

11. An endoscope, comprising an actuator for an intraductal moving body according to claim 7.
